(19) United States
(12) Patent Application Publication
(10) Pub. No.: US 2024/0287391 A1
(43) Pub. Date: Aug. 29, 2024

(54) HIGH TEMPERATURE PYROLYSIS OF HYDROCARBONS IN MOLTEN SALTS

(71) Applicant: Braskem America, Inc., Philadelphia, PA (US)

(72) Inventors: Samuel L. Leung, Pittsburgh, PA (US); Nicholas Wang, Pittsburgh, PA (US); Katherine McCullough, Pittsburgh, PA (US); Goutham Kothamreddy, Pittsburgh, PA (US); Charles Ambrass, Pittsburgh, PA (US); Matthew Feeney, Pittsburgh, PA (US); Jan Kalfus, Pittsburgh, PA (US); Scott Mitchell, Pittsburgh, PA (US)

(73) Assignee: Braskem America, Inc., Philadelphia, PA (US)

(21) Appl. No.: 18/432,900

(22) Filed: Feb. 5, 2024

Related U.S. Application Data
(60) Provisional application No. 63/483,559, filed on Feb. 7, 2023.

(51) Int. Cl. C10G 9/34

(52) U.S. Cl. CPC C10G 9/34; C10G 2300/10; C10G 2300/4006; C10G 2300/4012; C10G 2400/20; C10G 2400/22

(57) ABSTRACT

A process for the cracking of a carbon-containing feedstock to produce olefins, the process includes contacting, in a reactor system, the carbon-containing feedstock with a molten salt matrix consisting of a eutectic mixture of alkali metal carbonates, alkaline earth metal carbonates, or a mixture of any two or more thereof, to generate an olefin-containing product stream; and collecting an olefin from the olefin-containing product stream; wherein: the process is conducted in the absence of oxygen, and in the absence of a catalyst comprising a transition metal, a transition metal oxide, a rare-earth metal, a rare earth metal oxide, or a combination of any two or more thereof.

### CROSS-REFERENCE TO RELATED APPLICATIONS

This application claims the benefit of and priority to U.S. Provisional Application No. 63/483,559, filed Feb. 7, 2023, the contents of which are incorporated herein by reference in their entirety.

### FIELD

The present technology is generally related to the cracking of carbon-containing feedstocks. More specifically, it is related to methods of cracking carbon-containing feedstocks using a molten salt to form olefinic and aromatic monomers from a carbon-containing feedstock.

### BACKGROUND OF THE INVENTION

Pyrolysis is a promising technology that can be used to produce valuable oils and light hydrocarbons in high yields from waste plastic feedstocks. In this regard, ethylene and propylene are the key feedstocks for the production of polyolefins (i.e. polyethylene and polypropylene). The majority of ethylene and propylene is produced by steam cracking, a process in which a fossil-based, hydrocarbon feed (composed primarily of naphtha, liquified petroleum gas (LPG), and/or ethane) is diluted with steam and heated in a furnace in the absence of oxygen to temperatures around 850° C. This process is highly endothermic, typically requiring ˜60 GJ/ton of high value products. Conventional furnaces burn hydrogen (produced from the steam cracking process) and methane to meet this energy requirement, resulting in large emissions of CO₂. The continued dependence on both a fossil-based feedstock and fuel contributes greatly to the emissions of petrochemical operations.

Plastics account for roughly 12% of municipal solid waste in the U.S. and constitute a potential alternative/supplemental hydrocarbon feedstock. Specifically, polyolefins consist of long chains of carbons that, in atomic composition and arrangement, do not differ greatly from conventional fossil-based feedstocks, but do differ in average molecular weight (1000s of g/mol vs. 100-200 g/mol, respectively). Polyolefins, as a feedstock, are attractive because they present a potential opportunity for closed-loop circular plastics.

Waste plastics can contain a number of both organic and inorganic contaminants from both their production (e.g. additives, pigments, metals, etc.) and their subsequent processing (e.g. paper, PVC, water, etc.). Plastics thus cannot be easily used directly as a supplemental or alternative feedstock to a steam cracker for the production of light olefins.

Pyrolysis can be an intermediate processing step to convert polyolefins to pyrolysis oil (or “py-oil”). This process breaks down polyolefins into liquid “py-oil” fractions (i.e., naphtha, diesel), solids (waxes), and lower molecular weight gases. Char/carbon is often a low-value byproduct of the process. The naphtha can then be fed to a conventional cracker to produce light olefins. The process typically occurs around 500° C., as higher temperatures can lead to side reactions, including aromatization. Conventional pyrolysis, however, struggles with fouling (from char) and contaminants (e.g. metals, PVC), which can become entrained in the liquid products, exceeding limits imposed by the steam cracker. As a result, efforts are underway to improve purification of the feed plastic as well as the pyrolysis oil.

### SUMMARY

In one aspect, a process is provided for the cracking of a carbon-containing feedstock to produce olefins including contacting, in a reactor system, the carbon-containing feedstock with a molten salt matrix consisting of a eutectic mixture of alkali metal carbonates, alkaline earth metal carbonates, or a mixture of any two or more thereof, to generate an olefin-containing product stream; and collecting an olefin from the olefin-containing product stream. The processes described herein are conducted in the absence of oxygen, and in the absence of a catalyst comprising a transition metal, a transition metal oxide, a rare-earth metal, a rare earth metal oxide, or a combination of any two or more thereof. The eutectic mixture of alkali metal or alkaline earth metal carbonates may include carbonates of lithium (Li), sodium (Na), and potassium (K).

### BRIEF DESCRIPTION OF THE DRAWINGS

FIG. 1 is a graph of the mass yields at various temperature, according to the Examples, for three runs. As illustrated in FIG. 1, the cracking system efficiently consumes a model paraffin wax system and produces alpha olefin monomers in good yields that increase with increasing temperature to about 650° C.

FIG. 2 is a graph of the TGA curves in nitrogen of (dotted black) virgin HDPE and (dotted red) a mixture of 25 wt % HDPE and 75 wt % eutectic salt mixture, according to the Examples. The TGA curves are normalized to the amount of HDPE loaded and disregard the residual mass of the salt.

FIG. 3 is a graph showing the gas phase product distribution of the pyrolysis of PVC (blank) and pyrolysis of PVC in the presence of eutectic salt mixture (PVC+Eu), according to the Examples.

FIG. 4 is a XRD pattern of molten salt after reaction with PVC. The dashed lines indicate diffraction patterns corresponding to KCl and LiCl, according to the Examples. The remaining diffraction peaks correspond to carbonates.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and may be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “cracking” refers to a chemical process whereby a feedstock, i.e. complex organic molecules such as long-chain hydrocarbons, carbohydrates, or others are broken down into simpler molecules such as light hydrocarbons, oxygenates, or carbon oxides by the breaking of chemical bonds in the feedstock.

As used herein, the term “thermocracking” refers to a cracking process, whereby the conversion of feedstock to products is achieved by thermal energy transfer, i.e. heating, and, hence, it requires operating at elevated temperatures to proceed.

As used herein, the term “reactor system” refers to where the cracking reaction(s) take place. The process for cracking of hydrocarbons may occur in a single reactor or in at least two reactors in series.

As used herein, the term “carbon-containing feedstock” is not only purely hydrocarbon materials as would typically be associated with the term, but as long as there is a carbon-containing segment within a plastic (i.e. polymer) or biomass or biowaste that is amenable to cracking with the catalyst compositions provided herein, it meets the definition. For example, the carbon-containing feedstock may contain oxygen in the material, as well as other heteroatoms (N, P, S, Cl, etc.) and other materials such as fillers (including silica, zinc oxide, titanium oxide, calcium carbonate, etc.), colorants, plasticizers, and the like typically associated with polymers.

As used herein, the term a “eutectic” or “eutectic mixture” refers to a homogeneous mixture of substances that melt or solidify at a single temperature that is lower than the melting point of any of the constituents individually. It does not necessarily refer to the lowest melting point that is achievable with any particular mixture of substances, this is the eutectic point for those substances, and it may be part of the eutectic mixture. As long as a mixture of substances melts at a temperature lower than the melting point of any of its constituting pure substances, and forms a single continuous phase, it is a “eutectic” or “eutectic mixture” for the purposes of this disclosure. As used herein, the phrase “a eutectic mixture of alkali metal or alkaline earth metal, halides, (bi)carbonates, or hydroxides” may be alternatively recited as “a eutectic mixture of alkali metal carbonates, alkali metal hydroxides, alkaline earth metal carbonates, alkaline earth hydroxides, or a mixture of any two or more thereof.”

It has now been found that the cracking of carbon-containing feedstocks may be conducted over molten salt compositions in the absence of oxygen to form a product stream containing olefinic and/or aromatic compounds.

In this regard, the process according to the present invention presents an alternative means of implementing pyrolysis by incorporating a molten salt media. As used herein, molten salt is added as a kind of “processing agent”, thereby providing multiple benefits including improved heat transfer to the plastic particles as well as contaminant capture. The salt and trapped contaminants can potentially be purified later through physical (e.g. filtration) or chemical (e.g. re-oxidation) means.

The methods may be applied to carbon-containing feedstocks and includes petroleum feedstocks, recycling of olefinic polymers, biowaste, and biopolymers. The methods (or alternatively, processes) may be applied to pure hydrocarbon feedstock streams, to pure waste plastic streams (i.e. streams of only waste plastic), or to a mixture stream of hydrocarbon feedstock and waste plastic. The described methods have the potential to deliver improved performance over industry accepted methods such as thermal pyrolysis, thermal-steam cracking, fluid catalytic cracking, and supercritical fluid cracking. To assist in achieving a reaction in the absence of oxygen, the reaction may be conducted under an inert atmosphere. Illustrative inert gases include nitrogen, helium, and argon. Because the reaction is conducted in the absence of oxygen, there is little-to-no production of CO₂ as a byproduct when the carbon-containing feedstock is a non-oxygenated carbon-containing feedstock.

Additionally, the application of a molten salt is shown to improve heat transfer to the reaction and to aid in the capture of contaminants (e.g., HCl) while pyrolyzing waste plastic. Ultimately, these advancements look to improve the viability of pyrolysis for the conversion of plastics into chemicals.

In one aspect, a process for the cracking of a carbon-containing feedstock to produce olefins includes contacting, in a reactor system, the carbon-containing feedstock with a molten salt matrix consisting of a eutectic mixture of alkali metal carbonates, alkaline earth metal carbonates, or a mixture of any two or more thereof, to generate an olefin-containing product stream; and collecting an olefin from the olefin-containing product stream. The processes described herein are conducted in the absence of oxygen, and in the absence of a catalyst comprising a transition metal, a transition metal oxide, a rare-earth metal, a rare earth metal oxide, or a combination of any two or more thereof.

The processes described herein represent a process intensification, reducing the need for subsequent processing of product streams. Other accepted processes in the industry have multiple steps to obtain a desired product yield, greatly increasing the complexity of the process design.

The present methods do not require pressurization, instead being conducted from about 0.5 atmospheres (“atm”) to about 2 atm. In some embodiments, the process is carried out at ambient, atmospheric pressure. Alternative cracking processes, such as supercritical fluid cracking and high-pressure catalytic cracking, utilize much higher processing pressures, and, because of this, are more energy and capital-intensive processes.

The methods described herein also tolerate the presence of acid impurities, such as those containing chloride, bromide, sulfide, sulfate, and phosphate groups in the feed. The method also tolerates the presence of plastic filler components, such as silica, titania, zinc oxide, calcium carbonate, and others. The removal of such acid impurities and plastic filler components is believed to occur through the absorption of such materials into the eutectic mixture, which is then purified, and the impurities removed. Thus, the method is feedstock flexible and can be used to process mixed plastic waste. The methods may also be operated as a continuous, semi-continuous, or batch processes. The methods may also, in some embodiments, be carried out in a continuously stirred tank reactor. Thus, the method offers a high degree of flexibility for its end-user application design and operation. The carbon-containing feedstock may be injected into the reactor at either a bottom (i.e. it flows through the molten salt) or a top (i.e. it comes into contact at a surface of the molten salt) of the reactor.

As noted above, the eutectic mixture melts at a lower temperature than its constituent materials, and it melts at a temperature at which the catalytic reactions may be conducted to form desirable materials from a feedstock. The eutectic mixture of alkali metal carbonates may be a mixture of Li, Na, and K carbonates.

In particular embodiments, the molten matrix containing a eutectic mixture of carbonates of Li and Na in a mole ratio of about 52:48, respectively, or a eutectic mixture of carbonates of Li and K in a mole ratio of about 62:38, respectively, or a eutectic mixture of carbonates of Li, Na, and K, in a mole ratio of about 43.5:31.5:25, respectively.

In some embodiments, the eutectic mixture is one of Li₂CO₃, Na₂CO₃, and K₂CO₃. In some embodiments, the eutectic mixture of alkali metal carbonates has a melting point of less than about 800° C., or less than about 750° C., or less than about 650° C. This includes melting points from about 250° C. to about 650° C., from about 350° C. to about 550° C., or about 400° C. Other illustrative eutectic mixture of alkali metal carbonates includes those in Table 1, reproduced from Mutch et al. J. Mater. Chem. A 7, 12951-12973 (2019).

TABLE 1
Melting points of the individual alkali-carbonate
salt compounds and their eutectic mixtures.

| Salt System | Melting Point (° C.) |
|---|---|
| Li₂CO₃ | 723 |
| Na₂CO₃ | 854 |
| K₂CO₃ | 891 |
| Li₂CO₃—Na₂CO₃ (52-48 mol %) | 501 |
| Li₂CO₃—K₂CO₃ (62-38 mol %) | 498 |
| Na₂CO₃—K₂CO₃ (56-44 mol %) | 710 |
| Li₂CO₃—Na₂CO₃—K₂CO₃ (43.5-31.5-25 mol %) | 397 |
| Na₂CO₃—BaCO₃ (52.2-47.3 mol %) | 686 |

In the process, a carbon-containing feedstock is injected into a reactor system where it comes into contact with the molten salt. An upper temperature limit within the reactor is defined by the need to preserve a substantial fraction of the carbon-carbon bonds of the feed from the decomposition. For example, in an embodiment, the upper temperature limit is about 1100° C.

The increase in product yields with increasing temperature likely reflects the predominant reactions that take place during pyrolysis, specifically radical-mediated steps. Scheme 1 shows potential reaction pathways and propagation steps that lead to the formation of pyrolysis products. These reactions include a) initiation steps, in which a radical is initially formed by C-C cleavage, b) propagation steps, including b.i) b-scission, b.ii) H-shift, and b.iii) H-transfer steps, and c) termination steps, in which radicals are quenched. In radical-mediated reactions, the propagation steps b) occur at much faster rates than steps a) and c). As such, steps b) determine the product distribution and selectivity towards a given product, while steps a) and c) control the amount of product produced. Higher temperatures can help to stabilize and increase the lifetime of radical intermediates, thus increasing the number of propagation steps (steps b)) that can occur before they are quenched (step c)). These steps, particularly b-scission (step b.i)), form light olefins selectively. Thus, increasing temperatures is always expected to increase light olefin yield to that side reactions do not begin to consume products and reactants to a significant extent. Such reactions, which may directly form carbon deposits (e.g. methane pyrolysis), can potentially occur at temperatures above 1100° C.

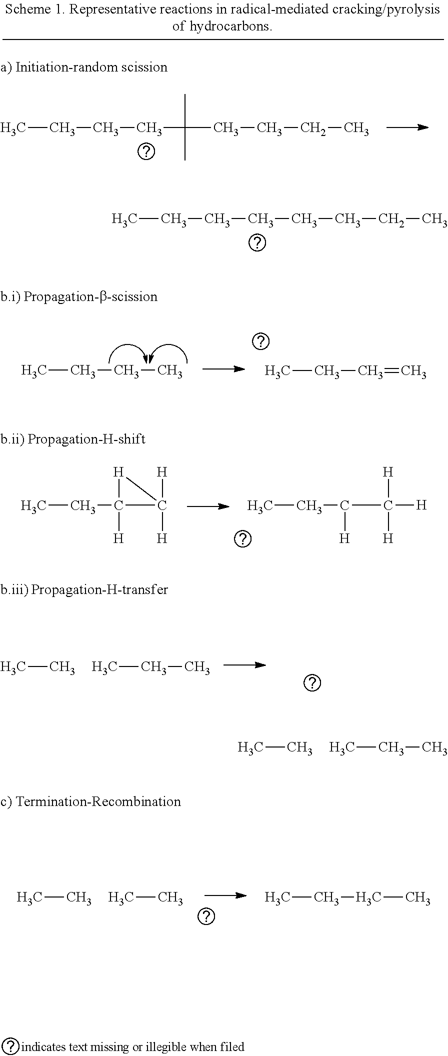

Processes designed for thermocracking of polyethylene can operate at a temperature exceeding the ceiling temperature by 50-150° C. in order to achieve economically practical feed-to-monomers conversion rates. In the case of other feeds, the upper temperature limit is a function of the ceiling temperatures of the corresponding monomers, as given in the values in Table 2, reproduced from Stevens, M. P. Polymer Chemistry an Introduction (3rd ed.). New York: Oxford University Press. pp. 193-194 (1999), plus the additional 50-150° C. to achieve practical rates,

TABLE 2
Ceiling temperatures of common hydrocarbon monomers.

| | Ceiling | |
|---|---|---|
| Monomer | Temperature (° C.) | Structure |
| ethylene | 610 | CH₂═CH₂ |
| 1,3-butadiene | 585 | CH₂═CHCH═CH₂ |
| isoprene | 466 | CH₂═C(Me)CH═CH₂ |
| styrene | 395 | PhCH═CH₂ |
| methyl methacrylate | 198 | CH₂═C(Me)CO₂Me |
| isobutylene | 175 | CH₂═CMe₂ |
| α-methylstyrene | 66 | PhC(Me)═CH₂ |

A lower temperature limit is defined by the need to maintain the molten salt in the liquid state. As an illustration, this is about 397° C. for the Li₂CO₃—Na₂CO₃—K₂CO₃ (43.5-31.5-25 mol %) eutectic mixture.

The process provides for the production of olefinic and/or aromatic compounds from a carbon-containing feedstock. Because the carbon-containing feedstock may be from a wide variety of materials for process, including the processing of recycled plastics, biomass, biowaste, mixed plastics, biomass, and/or biowaste, the olefinic and/or aromatic compounds that are produced may include a wide variety of unsaturated compounds such as, but not limited to light olefins, α-olefins, terminal dienes, substituted and unsubstituted aromatic compounds, including single aromatic ring or several aromatic ring compounds.

Illustrative olefinic and/or aromatic compounds are from a wide range of materials. In some embodiments, the olefinic compounds may be from C₂ to C₂₀ olefins, from C₂ to C₁₆ olefins, or from C₂ to C₁₂ olefins, or from subranges of any of these. In some embodiments, the aromatic compounds may be from C₆ to Cis aromatics, or from C₆ to C₁₂ aromatics, or from subranges of any of these. Illustrative olefinic and/or aromatic compounds include, but are not limited to, ethene, propene, 1-butene, 2-methyl-but-1-ene, 1-n-pentene, 2-methyl-pent-1-ene, 3-methyl-pent-1-ene, 1,3-butadiene, 1,3-pentadiene, 1,4-pentadiene, 1,3-hexadiene, 1,4-hexadiene, 1,5-hexadiene, benzene, toluene, ethylbenzene, xylenes, styrene, α-methylstyrene, naphthalene, and anthracene.

As noted herein, the carbon-containing feedstock may include, but is not limited to, any one or more of a refinery range hydrocarbons, a polymer, a biopolymer, biomass, or biowaste. Where the feedstock includes a polymer, illustrative polymers include, but are not limited to those such as polyethylene, polypropylene, polyisobutylene, polybutadiene, polystyrene, poly-α-methylstyrene, polacrylates, poly(meth)acrylates, polyvinyl acetate, and polyvinylchloride. Where the feedstock includes a biopolymer or other bio-based material it may include materials such as fatty acids, triglyceride esters of fatty acids, cellulose, lignin, sugars, animal fat, tissue, and ordure.

Refinery range hydrocarbons are typically defined by their boiling point range fractions. For example, light naphtha has an approximate boiling point range of 25 to 85° C. (gas phase or light organics), heavy naphtha has an approximate boiling point range of 85 to 200° C., kerosene has an approximate boiling point range of 170 to 265° C., gas oil has an approximate boiling point range of 175 to 345° C., and heavy residue has an approximate boiling point range of 345 to 656° C. All of these may serve as the feedstock for a refinery range hydrocarbon. In some embodiments where the feedstock includes a refinery range hydrocarbon, it may include asphalt, vacuum resid, heavy residual oil, paraffin wax, lubricating oil, diesel, kerosene, naphtha, or gasoline. In some embodiments, the feedstock may include n-hexane, n-hexadecane, or white mineral oil.

In the process, the temperature inside the reactor system is dependent upon the composition of the carbon containing feed and the desired reaction products. Accordingly, the temperature may be from the melting point of the eutectic mixture of alkali carbonates up to about 1000° C. This may include a temperature from about 250° C. to about 1000° C. In some embodiments, the contacting in the process is carried out at a temperature of less than 650° C. This may include, but is not limited to, a temperature of about 600° C. or less, such as about 450° C. to less than 650° C., or at about 600° C.

In the process, the pressures inside the reactor system may be low by comparison to other similar processes. For example, the pressure may be from about 0.5 atm to about 2 atm, preferably from 1 atm to about 2 atm, but it is preferably carried out at atmospheric pressure.

In the process, the residence time is a measure of how long the feedstock and products remain within the reaction on an average basis. In some embodiments, the residence time for the process is about 1 minute to about 15 minutes.

The present invention, thus generally described, will be understood more readily by reference to the following examples, which are provided by way of illustration and are not intended to be limiting of the present invention.

### EXAMPLES

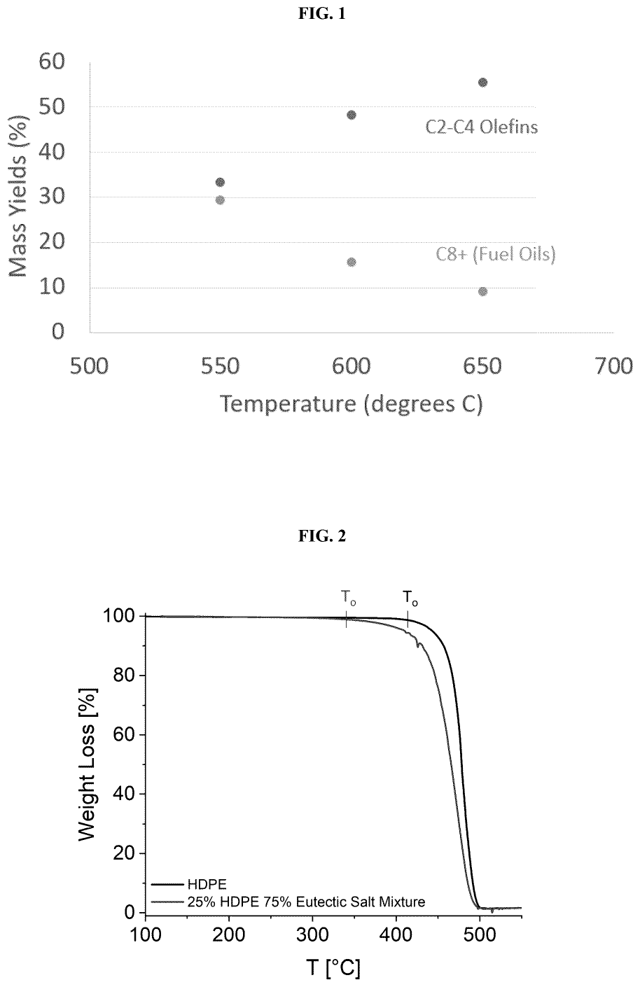

Example 1. Generation of olefinic monomers from paraffin waxes. Paraffin wax was melted at 90-120° C. and fed as a liquid onto a bed of molten salt (2 kg) (eutectic mixture of lithium, potassium, and sodium carbonate) contained in a lab-scale CSTR. The molten salt was maintained at temperature from about 550° C. to about 700° C. Optimal results were obtained at about 650° C. Feed rates of the paraffin wax were between 0.1-0.5 g/min. Nitrogen was introduced into the system at a total gas flow rate of 250-1000 mL/min. The contents of the CSTR were stirred using two impellers, one in the gas phase and one in the eutectic, each at 300 rpm. Olefin yields increased with increasing temperatures. FIG. 1 illustrates the results from three runs. As seen in this graph, the present cracking system efficiently consumes a model paraffin wax system and produces α-olefin monomers.

Alternatives. Paraffin waxes are used here as a model for heavy hydrocarbons and polyethylene feeds. They can be introduced presumably in any phase; with solids requiring additional energy input. The feed may be introduced from the bottom of the reactor to increase contact time. The reactor may be done in any configuration, including in a pipe (plug-flow reactor).

Example 2. Generation of olefinic monomers from different types of hydrocarbon feedstocks. The molten salt used consists of a eutectic mixture of Li₂CO₃ (32.1 wt %), K₂CO₃ (34.5 wt %), and Na₂CO₃ (33.4 wt %). Such a eutectic mixture minimizes the melting point (˜397° C.), enabling a wide range of operating temperatures between 400° C. and 800° C. Four types of hydrocarbon feedstocks were used: 1) hexadecane (nC16), 2) paraffin wax, 3) high density polyethylene (HDPE, MW: ˜75000 g mol⁻¹), 4) polypropylene (PP, MW: ˜220000 g mol⁻¹).

Continuous flow studies were conducted using a ˜5 L continuously stirred reactor (CSTR) containing 2 kg of the molten eutectic salt as described. Gas feeds (0.250-2 L min⁻¹) were introduced through a dip tube to the bottom of the molten salt bed, while liquid feeds were introduced using an ISCO syringe pump through either the same dip tube or through a secondary tube approaching the top of the molten salt bed. Polyolefin feeds (HDPE, PP) were introduced using the liquid dip tube by first heating the polymers in the ISCO syringe pump to 180° C. for HDPE and 220° C. for PP. The CSTR was heated using an electric furnace. All lines after the reactor were heated to a minimum temperature of 220° C. to prevent condensation of products. Product analysis and quantification were performed using gas chromatography.

Pyrolysis typically takes place at temperatures around 500° C. to prevent side reactions such as cyclization and aromatization, which form precursors to solid products including char and coke. However, these temperatures primarily yield liquid-range products that need to be processed further to yield light olefins. For these experimental tests, hexadecane (0.48 g min⁻¹), paraffin wax (0.1 g min⁻¹), and polymers (HDPE and PP; 0.48 g min⁻¹) were fed to the CSTR reactor as described in the absence of O₂ (0.95 L min⁻¹ N₂) to determine the effects of temperature on product yields.

Tables 3-6 show mass yields at temperatures between 500° C. and 700° C. for each feed, respectively.

Hexadecane was used here as a surrogate for a naphtha-like fossil-based feed. The obtained data shows that the product yields of light (C₅ and lower) species invariably increase with increasing temperature, though at the expense of some aromatics. Nevertheless, coke formed at rates below limits of detection and could only be found within the reactor after an extensive number of experiments. These deposits were not found within the molten salt itself, thus indicating that the molten salt may aid in the breakdown of hydrocarbons and thus prevent build-up of carbon on reaction vessels.

TABLE 3
Hexadecane (0.48 g min⁻¹) mass yields as a
function of temperature (500-700° C.)

| | Temperature (° C.) | | | | |
|---|---|---|---|---|---|
| Product | 500 | 520 | 550 | 600 | 650 |
| Methane | 0.3% | 1.4% | 2.0% | 8.9% | 14.9% |
| Ethane | 0.4% | 1.9% | 2.2% | 6.1% | 7.0% |
| Ethylene | 1.1% | 4.4% | 6.8% | 23.8% | 34.8% |
| Propane | 0.0% | 0.3% | 0.2% | 0.7% | 0.6% |
| Propylene | 0.8% | 3.7% | 5.5% | 17.6% | 17.2% |
| n-Butane | 0.0% | 0.1% | 0.1% | 0.1% | 0.1% |
| Butenes | 0.6% | 2.5% | 3.5% | 4.7% | 4.6% |
| Butadiene | 0.5% | 1.9% | 2.6% | 4.2% | 2.7% |
| Liquids/Solids (C5+) | 96.2% | 83.7% | 56.3% | 30.5% | 8.3% |
| Aromatics | 0.1% | 0.1% | 0.2% | 3.4% | 9.8% |

TABLE 4
Paraffin wax (0.1 g min⁻¹) mass yields as a
function of temperature (550-700° C.)

| | Temperature (° C.) | | | |
|---|---|---|---|---|
| Product | 550 | 600 | 650 | 700 |
| Methane | 0.3% | 1.4% | 7.5% | 15.8% |
| Ethane | 0.4% | 1.2% | 3.1% | 4.6% |
| Ethylene | 1.6% | 7.1% | 24.0% | 40.8% |
| Propane | 0.0% | 0.1% | 0.3% | 0.4% |
| Propylene | 1.3% | 6.2% | 16.9% | 17.7% |
| n-Butane | 0.0% | 0.1% | 0.1% | 0.1% |
| Butenes | 0.6% | 2.2% | 4.1% | 2.6% |
| Butadiene | 0.0% | 0.1% | 0.0% | 0.0% |
| Liquids/Solids (C5+) | 95.6% | 78.8% | 56.3% | 10.8% |
| Aromatics | 0.2% | 2.8% | 8.4% | 7.2% |

TABLE 5
HDPE-A (0.48 g min⁻¹) mass yields as a function
of temperature (550-650° C.)

| | Temperature (° C.) | | |
|---|---|---|---|
| Product | 550 | 600 | 650 |
| Methane | 0.4% | 1.8% | 4.9% |
| Ethane | 0.7% | 1.7% | 3.0% |
| Ethylene | 1.5% | 5.8% | 15.1% |
| Propane | 0.6% | 0.7% | 0.8% |
| Propylene | 1.8% | 6.1% | 13.9% |
| n-Butane | 0.5% | 0.4% | 0.4% |
| Butenes | 1.6% | 4.6% | 8.6% |
| Butadiene | 0.9% | 2.3% | 4.0% |
| Liquids/Solids (C5+) | 91.6% | 73.6% | 43.7% |
| Aromatics | 0.4% | 3.0% | 5.6% |

TABLE 6
PP (0.48 g min⁻¹) mass yields as a function
of temperature (550-650° C.)

| | Temperature (° C.) | | |
|---|---|---|---|
| Product | 550 | 600 | 650 |
| Methane | 1.5% | 4.4% | 7.9% |
| Ethane | 2.2% | 4.1% | 4.4% |
| Ethylene | 2.1% | 5.0% | 8.8% |
| Propane | 0.6% | 1.3% | 1.0% |
| Propylene | 10.4% | 16.7% | 16.2% |
| n-Butane | 0.2% | 0.3% | 0.2% |
| Butenes | 6.8% | 12.5% | 12.8% |
| Butadiene | 0.6% | 1.2% | 1.3% |
| Liquids/Solids (C5+) | 72.4% | 47.4% | 36.4% |
| Aromatics | 3.2% | 7.1% | 11.0% |

Example 3. Thermogravimetric analysis (TGA) was performed using a Mettler Toledo TGA/DSC 3+. Analysis was performed on virgin high density polyethylene (HDPE), and a mixture containing 25 wt % of virgin HDPE and 75 wt % of the molten salt mixture. Samples were placed in sapphire crucibles at atmospheric pressure and heated in nitrogen at 10 mL min⁻¹. The following heating procedure was performed for each sample: 1) heat from 25° C. to 120° C. at a rate of 10° C./min, 2) hold at 120° C. for 30 minutes in order to remove physisorbed water, 3) heat to 600° C. at 10° C./min, and 4) hold for 30 minutes.

TGA profiles were collected with and without a eutectic molten salt mixture. FIG. 2 illustrates the TGA profile for pure HDPE (black line in FIG. 2) and 25 wt % of HDPE in 75 wt % of eutectic molten salt mixture (red line in FIG. 2) under nitrogen flow. Weight loss corresponds to the decomposition of the HDPE polymer. The onset temperature (Tₒ) for mass loss is defined as the temperature in which 1% mass is loss. In nitrogen, HDPE begins to decompose at 414° C. and is completely decomposed by 500° C. The effect of the addition of eutectic molten salt mixture is readily seen (FIG. 2); Tₒ is decreased to 330° C. with the addition of the salt, and the rate of decomposition is also greatly accelerated. These data indicate that the salt aids in the breakdown of the polymer. The shift to lower decomposition temperature in the presence of the molten salt mixture may indicate improved heat transfer to the HDPE (and which occurs even in the absence of oxygen). We can deduce that accelerated heat transfer from the molten salt to the polymer is key for lowering the decomposition temperature and accelerating the rate at which it decomposes.

Example 4. The ability of the molten salt mixture to uptake chlorine contamination was studied by running pyrolysis reactions on pure PVC and PVC in the presence of a eutectic salt mixture. Reactions were run in a batch phase system under nitrogen at 450° C. for five minutes. The headspace was analyzed using Gas chromatography-mass spectrometry (GC-MS) and the gas phase products that are volatile up to 130° C. were analyzed. Powder x-ray diffraction patterns of the spent salt were collected to determine the presence of salt phases and compounds.

Regarding the contaminant-capturing effects of molten salts, decomposition of PVC occurs in two stages: the first occurs at low temperature where the chlorine is removed from the polymer backbone, and the second at higher temperature, where the polymer chain then decomposes. After the removal of chlorine, a hydrogen is abstracted from the polymer chain resulting in the formation of HCl. This also increases the tendency for the polymer to aromatize to produce benzene.

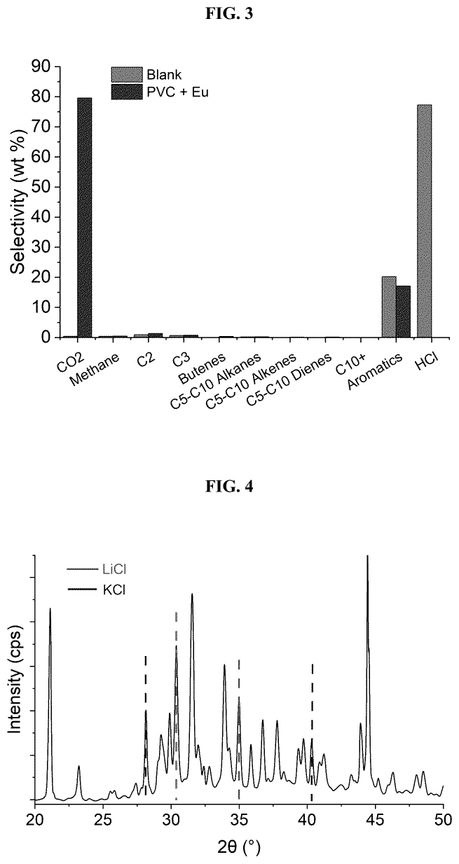

The ability of the salt to uptake chlorine contamination was studied by running pyrolysis reactions on pure PVC and PVC in the presence of a eutectic salt mixture. The gas phase product distributions are shown in FIG. 3. These are the two major products in PVC pyrolysis in nitrogen. When the eutectic salt is added to the PVC, the major products become benzene and CO₂ with no selectivity towards HCl. This indicates that the salt is reactive in the presence of chlorine, exchanging carbonate ions with chlorine to form chloride salts and CO₂ as a byproduct. This is confirmed by the XRD pattern of the salt after it has been exposed to PVC, shown in FIG. 4, Diffraction peaks corresponding to KCl and LiCl can be seen (FIG. 4) with no evidence of K₂CO₃ present after reaction. However, diffraction peaks related to Li₂CO₃ are still present indicating that only parts of the salt react with chlorine.

The experimental data demonstrates that a molten salt can be used to aid in the processing of polymer feeds by improving heat transfer capabilities while also capturing some of these contaminants. Such advancements enable pyrolysis to be a more viable technology for the preprocessing or single-step processing of waste plastics to high value chemicals.

While certain embodiments have been illustrated and described, it should be understood that changes and modifications may be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations may be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range may be easily recognized as sufficiently describing and enabling the same range being broken down into at least equal halves, thirds, quarters, fifths, tenths, etc. As a non-limiting example, each range discussed herein may be readily broken down into a lower third, middle third and upper third, etc. As will also be understood by one skilled in the art all language such as “up to,” “at least,” “greater than,” “less than,” and the like, include the number recited and refer to ranges which may be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A process for the cracking of a carbon-containing feedstock to produce olefins, the process comprising:
contacting, in a reactor system, the carbon-containing feedstock with a molten salt matrix consisting of a eutectic mixture of alkali metal carbonates, alkaline earth metal carbonates, or a mixture of any two or more thereof, to generate an olefin-containing product stream; and
collecting an olefin from the olefin-containing product stream;
wherein:
the process is conducted in the absence of oxygen, and in the absence of a catalyst comprising a transition metal, a transition metal oxide, a rare-earth metal, a rare earth metal oxide, or a combination of any two or more thereof.

2. The process of claim 1, wherein the eutectic mixture of alkali metal or alkaline earth metal carbonates comprises a mixture of Li, Na, and K carbonates.

3. The process of claim 1 that is a continuous process, a semi-continuous process, or a batch process.

4. The process of claim 1, wherein the reactor system comprises a continuously stirred reactor.

5. The process of claim 1, wherein the olefin comprises a C₂-C₁₂ olefin.

6. The process of claim 1, wherein the olefin comprises ethylene, propylene, 1-butene, 2-methyl-but-1-ene, 1-n-pentene, 1-n-hexene, 2-methyl-pent-1-ene, 3-methyl-pent-1-ene, 1,3-butadiene, 1,3-pentadiene, 1,4-pentadiene, 1,3-hexadiene, 1,4-hexadiene, 1,5-hexadiene, benzene, toluene, ethylbenzene, xylenes, styrene, α-methylstyrene, naphthalene, anthracene, or a combination of any two or more thereof.

7. The process of claim 1, wherein the olefin comprises ethylene or propylene.

8. The process of claim 1, wherein the contacting is conducted at a temperature of less than 800° C.

9. The process of claim 1, wherein the contacting is conducted at a temperature of less than 650° C.

10. The process of claim 9, wherein the contacting is conducted at a temperature of about 600° C. or less.

11. The process of claim 9, wherein the contacting is conducted at a temperature of about 450° C. to less than 650° C.

12. The process of claim 9, wherein the contacting is conducted at a temperature of about 600° C.

13. The process of claim 1, wherein the carbon-containing feedstock is fed to a bottom of the reactor system.

14. The process of claim 1, wherein the carbon-containing feedstock is fed to a top of the reactor system.

15. The process of claim 1 that is carried out at a pressure of about 0.5 atm to 2atm.

16. The process of claim 1 that is carried out at a pressure of about 1 atm.

17. The process of claim 1, wherein the carbon-containing feedstock comprises polyethylene, polypropylene, polyisobutylene, polybutadiene, polystyrene, poly-α-methylstyrene, polacrylates, poly(meth)acrylates, polyvinylchloride, or polyethylene terephthalate.

18. The process of claim 1, wherein the carbon-containing feedstock comprises a refinery range hydrocarbon.

19. The process of claim 17, wherein the refinery range hydrocarbon comprises asphalt, vacuum resid, heavy residual oil, paraffin wax, pyrolysis wax, lubricating oil, diesel, kerosene, naphtha, gasoline, or a combination of any two or more thereof.

20. The process of claim 17, wherein the refinery range hydrocarbon comprises a gas phase feed comprising at least one of methane, ethane, ethylene, propane, propylene, butane, isobutane, 1-butene, 2-butene, 1,3-butadiene, and hydrogen.

21. The process of claim 1 that is conducted in the presence of an inert gas.

22. The process of claim 21, wherein the inert gas comprises nitrogen, helium, or argon.
